(19) United States
(12) Patent Application Publication
(10) Pub. No.: US 2015/0175965 A1
(43) Pub. Date: Jun. 25, 2015

(54) NOVEL METHODS TO REGENERATE HUMAN LIMBAL STEM CELLS

(71) Applicant: THE REGENTS OF THE UNIVERSITY OF CALIFORNIA, Oakland, CA (US)

(72) Inventors: Sophie Xiaohui Deng, Los Angeles, CA (US); Hua Mei, Los Angeles, CA (US); Martin Nakatsu, Orange, CA (US); Sheyla Gonzalez, Los Angeles, CA (US)

(73) Assignee: THE REGENTS OF THE UNIVERSITY OF CALIFORNIA, Oakland, CA (US)

(21) Appl. No.: 14/405,890

(22) PCT Filed: Dec. 12, 2013
(86) PCT No.: PCT/US2013/044375

§ 371 Date: Dec. 5, 2014

Related U.S. Application Data
(60) Provisional application No. 61/655,836, filed on Jun. 5, 2012.

(51) Int. Cl. C12N 5/0797; C12M 1/00; C12M 1/12

(52) U.S. Cl. CPC C12N 5/0623; C12M 25/02; C12M 23/34; C12N 2506/09; C12N 2502/085

(57) ABSTRACT

The invention disclosed herein provides systems and methods designed to facilitate human limbal stem/progenitor cell culture including a novel 3-dimensional (3D) sandwich method/system in which human limbal stem/progenitor cells and feeder cells are separately cultured on opposite sides of a porous membrane.

### CROSS-REFERENCE TO RELATED APPLICATIONS

This application claims the benefit under 35 U.S.C. Section 119(e) of co-pending U.S. Provisional Patent Application Ser. No. 61/655,836 filed Jun. 5, 2012, the contents of which are incorporated herein by reference.

### STATEMENT AS TO RIGHTS TO INVENTIONS MADE UNDER FEDERALLY SPONSORED RESEARCH AND DEVELOPMENT

This work was supported by NEI R01EY021797 and 2P30E4000331-44 from the National Eye Institute. The Federal Government has certain rights in this invention.

### TECHNICAL FIELD OF THE INVENTION

The present invention relates to systems and methods for cultivating corneal epithelial stem cells.

### BACKGROUND OF INVENTION

Corneal epithelial stem cells, also referred to as limbal stem cells (LSCs), are found at the basal layer of limbal epithelium of the cornea (see, e.g. Ebato et al., Invest Ophthalmol Vis Sci. 28, 1450, 1987; Huang et al., Invest Ophthalmol Vis Sci. 32, 96, 1991; Tsai et al., Ophthalmology 97, 446, 1990; Cotsarelis et al., Cell 57, 201, 1989; and Davanger et al., Nature. 229, 560, 1971). When the LSCs are deficient and unable to repopulate the corneal surface, the cornea surface will become opaque. Limbal stem cell deficiency (LSCD) in patients causes inflammation, vascularization, scarring, pain, and ultimately blindness (see, e.g. Tseng, S. C. Eye. 3 (Pt 2), 141, 1989; Sejpal et al., Middle East Afr J Ophthalmol. 20, 5, 2013; and Dua et al., Indian J Ophthalmol. 48, 83, 2000).

Transplantation of ex vivo expanded LSCs can successfully restore vision in many affected individuals (see, e.g. Pellegrini et al., Lancet. 349, 990, 1997; and Rama et al., The New England journal of medicine. 363, 147, 2010). Currently, the standard method for propagating LSCs in vitro is to culture the LSCs directly on growth-arrested mouse fibroblast 3T3 feeder cells. Cultured stem/progenitor cells form 2-dimensional (2D) colonies that expand and push away the feeder cells.

A number of challenges are associated with the current 2D culture methods. One is the varying distances between the cultured cells and the feeder cells. Feeder cells support the ex vivo expansion of LSCs by secreting soluble niche molecules, including growth factors and cytokines, and probably also by signaling through cell-cell contact (see, e.g. Miyashita, et al., Tissue Eng Part A. 14, 1275, 2008). Because of the distance between the center of colonies and the feeder cells, gradients of nutrients form. Stem cell markers such as N-cadherin, p63α, and ABCG2, are expressed at higher levels at the edge of the colonies, while the expression of the differentiation marker K12 is greatest near the center of the colonies (see, e.g. Secker, G. A., and Daniels, J. T. Limbal epithelial stem cells of the cornea. 2008; and Higa et al., Invest Ophthalmol Vis Sci. 50, 4640, 2009). These observations provide evidence that the close proximity to feeder cells helps in maintaining the less differentiated state of LSCs.

Another shortcoming of standard 2D culture methods is the competition between the stem cells and the feeder cells for the growth surface. As the epithelial colonies grow, they push away the feeder cells; this can result in a progressive decrease in the number of feeder cells in culture, which can lead to an insufficient supply of nutrients for the LSCs. Another issue with standard 2D culture methods is the possible contamination by murine feeder cells. Specifically, because of the direct contact between the LSCs and the feeder cells, it is possible that not all feeder cells are removed from the LSC population after harvest. Thus, feeder cells are a potential cross-contamination risk in clinical applications.

To mimic the in vivo environment of LSCs and to improve the current 2D culture method, various 3D methods to culture LSCs in vitro have been examined. LSCs are presumed to be in close proximity with their niche cells. LSCs and their subjacent mesenchymal niche cells have been isolated by collagenase treatment and co-cultured in a 3D matrigel to form cell spheres (see, e.g. Xie et al., Invest Ophthalmol Vis Sci. 53, 279, 2012). However, the cell proliferation rate was not optimal and the percentage of epithelial cells in the cell spheres after culture was not known. Efforts have also been made to culture in vitro propagated limbal epithelial cells on top of the corneal stromal cells embedded either in collagen or in a fibrin matrix (see, e.g. Bray et al., Biomaterials. 33, 3529, 2012; and Papini et al., Differentiation. 73, 61, 2005). Unfortunately, the expansion rate and epithelial stem cell phenotypes after this type of 3D culture are unknown.

Culture systems and methods that can overcome the above-mentioned challenges of the current culture methods are desirable.

### SUMMARY OF THE INVENTION

Embodiments of the invention disclosed herein provide systems and methods designed to facilitate human limbal stem/progenitor cell culture including a novel 3-dimensional (3D) sandwich method in which limbal stem cells and feeder cells are separately cultured on opposite sides of a porous membrane. The systems and methods disclosed herein are useful to produce expanded populations of limbal stem cells derived from tissue explants. Limbal stem cells grown under these culture conditions exhibit a small, compact, cuboidal stem-cell morphology and other stem cell characteristics that comparable to those observed in limbal stem cells produced from standard culture methods. Moreover, limbal epithelial cell clusters cultured with the sandwich method are observed to have a significantly higher proliferation rate than those cultured with existing methods. Aspects of the invention include using the systems disclosed herein in a variety of cell culture methods including, for example, methods of generating cells having a human limbal stem cell phenotype.

The invention disclosed herein has a number of aspects. One aspect of the invention is a system for culturing human limbal stem cells of the corneal epithelium. Typically this system comprises a container comprising a culture media for the human limbal stem cells and a porous membrane disposed in the culture media so as to form a first cell culture compartment and a second cell culture compartment. The porous membranes used in these systems are designed to allow soluble factors to migrate between the first cell culture compartment and the second cell culture compartment while simultaneously preventing cells from migrating between the first cell culture compartment and the second cell culture compartment. In this system, human limbal stem cells grown in the first cell culture compartment and human feeder cells are growth in the second cell culture compartment, so that the human feeder cells secrete one or more soluble factors that faciliate the growth of the human limbal stem cells. As discussed in detail below, this cell culture system can be used to generate human limbal stem cells, to maintain human limbal cell phenotypes in culture, and to faciliate human limbal stem cell proliferation.

Embodiments of invention include systems designed to maintain human limbal stem cells in a selected polarity such as an apical-basolateral polarity. In illustrative embodiments of the invention, the first cell culture compartment and the second cell culture compartment are arranged in the system so that the human feeder cells are below the human limbal stem cells. Typically, for example, the porous membrane is disposed is the system in a horizontal orientation with the human limbal stem cells above, and the feeder cells below this horizontal membrane. In typical embodiments of the invention, the porous membrane comprises pores having a size less than 3 μm. Optionally the membrane is formed from material comprising a polyethylene terephthalate.

In certain embodiments of the invention, the human limbal stem cells used in the systems and methods of the invention are mechanicall or enzymatically processed prior to being placed into a cell culture system of the invention. In the working embodiment of the invention that are disclosed herein, prior to being placed in the first cell culture compartment these cells are scraped from limbal tissue, pretreated with a protease, and pipetted so as to break cell sheets into clusters of cells. In some embodiments of the invention, the human limbal stem cells used in the systems and methods of the invention are also observed in order to identify the presence or expression level of one or more biomarkers. In the working embodiment of the invention that are disclosed herein, prior to being placed in the first cell culture compartment these cells are for example, examined to observe p63α expression levels.

A related aspect of the invention is a system for culturing human limbal stem cells comprising a container comprising a culture media for human limbal stem cells, a limbal tissue sample explant disposed in the culture media wherein the tissue sample explant comprises human limbal stem cells; and human feeder cells disposed in the culture media at a location proximal to the human limbal stem cells so that soluble factors produced by the feeder cells migrate to the human limbal stem cells. In some embodiments of the invention, this system includes a porous membrane disposed in the culture media so as to form a first cell culture compartment and a second cell culture compartment, wherein the porous membrane allows soluble factors to migrate between the first cell culture compartment and the second cell culture compartment while simultaneously preventing cells from migrating between the first cell culture compartment and the second cell culture compartment.

Another aspect of the invention is a method of maintaining human limbal stem cells in an undifferentiated human limbal stem cell phenotype (e.g. a small, uniform and compact cellular morpology) by culturing the human limbal stem cell cells in the systems disclosed herein. Yet another aspect of the invention is a method of facilitating the proliferation of human limbal stem cells within cell clusters, the method comprising culturing the human limbal stem cells in the systems disclosed herein. Typically in these methods, the human limbal stem cells are disposed in the system at a location and in an orientation selected to control the polarity of these cells.

Additional aspects of the invention include methods of generating cells having a human limbal stem cell phenotype. Typically, such methods include disposing at least one of hair follicle stem cells, skin epithelial stem cells, embyronic stem cells or induced pluripotent stem cells in the a cell culture compartment of the systems disclosed herein. Such methods also include disposing at least one of disposing at least one of human limbal stromal cells or corneal stromal cells in a second cell culture compartment of the systems disclosed herein. In these methods, soluble factors to migrate from the cells in the second cell culture compartment to the first cell culture compartment, wherein the migration of the soluble factors results in the generation of human limbal stem cells from the hair follicle stem cells, epidermal stem cells, embyronic stem cells or induced pluripotent stem cells, so that human limbal stem cells are generated. In an illustrative embodiment of the invention, skin epithelial stem cells are disposed in the first cell culture compartment and human limbal stromal cells are disposed in the second cell culture compartment and factors produced by the limbal stromal cells modulate the differentiation of the skin epithelial stem cells in a manner that generates cells having a human limbal stem phenotype.

Other objects, features and advantages of the present invention will become apparent to those skilled in the art from the following detailed description including the Appendices. It is to be understood, however, that the detailed description and specific examples, while indicating some embodiments of the present invention are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1: Diagram illustrating standard and sandwich culture methods. In the standard culture method, limbal epithelial cells were cultured directly on feeder cells. In the sandwich culture method, feeder cells attached to the bottom of the PET membrane, and the cultured cells were seeded onto the inner side of the membrane.

FIG. 2: LSCs cultured in single-cell suspension. (A) Morphology of LSC colonies. (B) Proliferation rate of limbal epithelial cells. (C) Relative mRNA levels of putative stem cell markers and maturation markers as evaluated by qRT-PCR. The expression of markers from single-cell cultured using the standard method served as a control and was designated a value of 1. Asterisks indicate p<0.5 in comparison with results for the standard method.

FIG. 3: Comparison of LSCs derived from cell cluster standard and sandwich cultures. (A) Morphology of colonies. (B) Proliferation rate of limbal epithelial cells. (C) Relative mRNA levels of the putative stem cell markers and maturation marker as evaluated by qRT-PCR. The expression of markers by control single-cell standard culture served as a control and was designated a value of 1. Asterisks indicate p<0.5 in comparison with results for the cluster standard method or the control method. Ctl: control. CST: cluster standard culture method. CSW: cluster sandwich culture method.

FIG. 4: Expression of p63α, K14, and K12 in limbal epithelial cells derived from cluster standard and sandwich methods. (A) Representative images of p63α expression, and percentage and absolute number of p63α^{br} cells generated from cultured limbal cell sheets. (B) Representative images of K14 expression, and percentage and absolute number of K14⁺ cells generated from cell cluster cultures. (C) Representative images of K12 expression, and percentage and absolute number of K12⁺ cells generated from cell cluster cultures. The absolute number of cells was the total number of cells obtained from the culture divided by the number of cells seeded. Ctl: single-cell control. CST: cluster standard culture method. CSW: cluster sandwich culture method. Scale bar represents a distance of 100 μm.

FIG. 5: Limbal epithelial cells expanded from tissue explant cultures using the standard and sandwich methods. (A) Morphology of cells from explant outgrowth. (B) Relative rate of cell outgrowth. The cell outgrowth rate was calculated as the number of cells harvested per piece of explant. (C) The relative mRNA expression levels of putative stem cell markers and maturation maker of the outgrowth. EST: explant standard culture method. ESW: explant sandwich culture method.

FIG. 6: Expression of p63α, K14 and K12 in cell outgrowth from limbal explants in standard and sandwich cultures. (A) Representative images of p63α expression, and percentage and absolute number of p63α^{br} cells in the outgrowth of explants. (B) Representative images of K14 expression, percentage and absolute number of K14⁺ cells in the outgrowth of explants. (C) Representative images of K12 expression, and percentage and absolute number of K12⁺ cells in the outgrowth of explants. The absolute number of cells was the total number of cells obtained from culture divided by the number of cells seeded. EST: explant standard culture method. ESW: explant sandwich culture method. Scale bar represents a distance of 100 μm.

FIG. 7: Schematic of aspects of the invention. This figure provides illustrative embodiments including those using single cells, those using tissue explants and those using sheets.

FIG. 8: Limbal stem cell cultures. (A) Colony formation on NIH 3T3 feeder layer after 14 days of culture. The largest round colonies are holoclones that are presumed to be derived from stem cells. (B) Highly compacted cuboidal epithelial cells in a holoclone colony. (C) The cultures contained 9% of p63-bright cells (“p63-bright cells” are those holoclone-forming stem cells that stain intensely, see, e.g. Rama et al., N Engl J Med 2010; 363:147-55).

FIG. 9: Human epidermal epithelial progenitor cells. (A) Epidermal epithelial progenitor cells isolated from HF formed compact colonies on mouse 3T3 feeder cells. Progenitor phenotype of the epithelial cells were maintained after two passages in low Ca2+ condition. They expressed high level K15 (B) and p63α (C).

FIG. 10: Wnt6 maintains the stem/progenitor cell phenotype of LSCs. (A) Wnt6 was overexpressed in 3T3 cells. (B) Higher expression of stem cell markers and lower expression of K12 were observed in the LSCs cultured on Wnt6-overxpressing 3T3 feeder cells. Confirmation of overexpression of Wnt6 at the mRNA level.

FIG. 11(A): Expression of activated Notch1 in human corneal tissue. Activated Notch1 was detected in the basal and suprabasal limbal epithelial cells (lower panel). There were much fewer activated Notch1+ epithelial cells in the central cornea (upper panel). FIG. 11(B). The Notch ligand Dll1 immobilized on culture plates activates Notch signaling. C2C12 myoblasts transfected with a Notch luciferase reporter were cultured on immobilized Dll1Fc or Fc control or cocultured with Dll1-expressing or parental Ltk− cells prior to measuring luciferase activity. Activation of Notch reporter activity by Dll1Fc or Ltk-Dll1 cells is expressed relative to Fc or Ltk− cells, respectively.

FIG. 12: Stepwise schematic of an illustrative reprogramming co-culture protocol embodiment of the invention. Abbreviation: CSC, cornea stromal cells.

### DETAILED DESCRIPTION OF THE INVENTION

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

The present invention relates to systems and methods for cultivating human corneal epithelial stem cells, cells that are also called limbal stem cells (LSCs). A known method for cultivating human limbal stem cells is to grow these cells directly on top of feeder cells in culture. There a number of problems with this technology. For example, when colonies grow bigger, it is frequently observed that the cells at the center of colonies differentiate (an undesirable event), most likely due to the lack of nutritional support from distant feeder cells. In addition, it is difficult to isolate a pure population of these LSCs after culture due to the close contact with the feeder cells. The potential contamination of animal cells and products further limits clinical use of the human LSCs expanded via this method.

Embodiments of the present invention involve new systems and methods for cultivating human limbal stem cells (LSCs). The systems and methods disclosed herein can be used to expand human LSCs in culture to use for transplantation to treat limbal stem cell deficiency in humans. Embodiments of the invention can use cell sheets to culture human LSCs. In such embodiments of the invention the method includes the use of different types of human feeder cells including but not limited to the types of human feeder cells described herein. In illustrative embodiments of the invention, the method for cultivating human LSCs using a cell sheet method comprises removing Iris, endothelium, Tenon's capsule and conjunctiva from fresh human sclerocornea tissue. This embodiment further comprises the steps of incubating the tissue with dispase for 1-2 hours at 37° C. and separating epithelial cells from the stroma. The cells are then washed with growth media prior to being placed in a culture system as disclosed herein.

The 3D sandwich methods permit complete separation between cultured cells and feeder cells while providing an even and maximal proximity between them. These methods also permit the culturing of LSCs without the risk of feeder cell contamination. Moreover, data from the working examples disclosed herein shows that the methods disclosed herein produce cells exhibiting the morphology of the epithelial stem cell phenotype. This data further confirms that limbal fibroplasts, human bone marrow-derived mesenchymal stem cells and human adipose-derived mesenchymal stem cells support the growth of limbal epithelial cells as rapidly as 3T3 cells in various cell sheet methods. These methods further maintain the stem cell phenotype of limbal epithelial cells in a manner akin to that provided by the 3T3 cells for cell sheet methods.

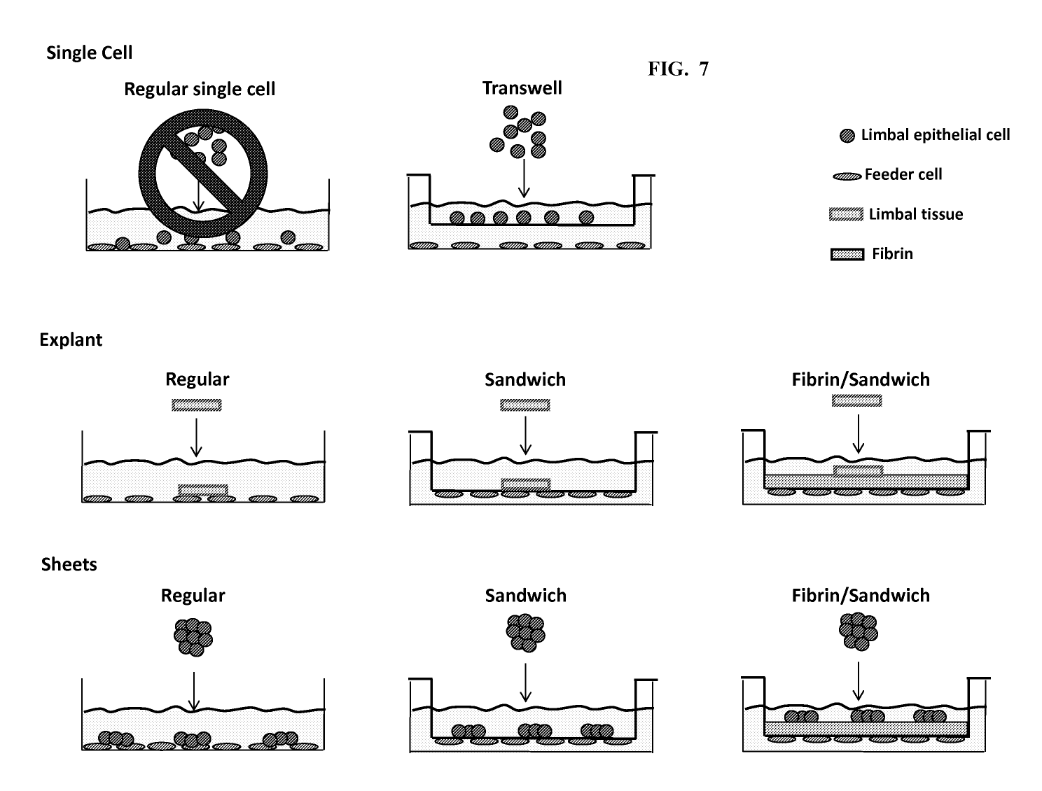

One aspect of the invention is a system for culturing human limbal stem cells of the corneal epithelium (embodiments of which are shown in FIG. 7). Typically this system comprises a container (100) comprising a culture media (200) for the human limbal stem cells (300) and a porous membrane (400) disposed in the culture media so as to form a first cell culture compartment (500) and a second cell culture compartment (600). The porous membranes used in these systems are designed to allow soluble factors such as those produced by the feeder cells (700) to migrate between the first cell culture compartment and the second cell culture compartment while simultaneously preventing cells from migrating between the first cell culture compartment and the second cell culture compartment. Embodiments of the invention include additional materials useful to grow these cells, for example a sheet of a fibrin material (800). In this system, human limbal stem cells grown in the first cell culture compartment and mammalian feeder cells are growth in the second cell culture compartment, so that the mammalian feeder cells secrete one or more soluble factors that faciliate the growth of the human limbal stem cells. As discussed in detail below, this cell culture system can be used to generate human limbal stem cells, to maintain human limbal cell phenotypes in culture, and to faciliate human limbal stem cell proliferation.

Many adult stem cells are designed to divide asymmetrically in vivo in order to balance self-renewal and differentiation, thereby maintaining tissue homeostasis. Asymmetric stem cell divisions depend on asymmetric cell architecture (i.e., cell polarity) within the cell and/or the cellular environment. A number of stem cells are polarized within their microenvironment, or the stem cell niche, and their asymmetric division relies on their relationship with the microenvironment. In such contexts, embodiments of invention include systems that maintain human limbal stem cells in a selected polarity such as an apical-basolateral polarity. In illustrative embodiments of the invention, the first cell culture compartment and the second cell culture compartment are arranged in the system so that the mammalian feeder cells are below the human limbal stem cells. Typically, for example, the porous membrane is disposed is the system in a horizontal orientation with the human limbal stem cells above, and the feeder cells below this horizontal membrane. In common embodiments of the invention, the porous membrane comprises pores having a size less than 3 μm. Optionally the membrane is formed from material comprising a polyethylene terephthalate.

In typical embodiments of the invention, the mammalian feeder cells used with the human limbal stem cells comprise human bone marrow derived mesenchymal stem cells, human adipose derived mesenchymal stem cells, or human limbal fibroblasts. In some embodiments of the invention, the mammalian feeder cells used with the human limbal stem cells comprise murine 3T3 cells. In embodiments of the invention, the feeder cells have been treated with mytomycin C or radiation so as to induce growth arrest. In certain embodiments of the invention, the human limbal stem cells used in the systems and methods of the invention are processed prior to being placed into a cell culture system of the invention. In the illustrative embodiment of the invention that are disclosed herein, prior to being placed in the first cell culture compartment these cells are scraped from limbal tissue, pretreated with a protease, and pipetted so as to break cell sheets into clusters of cells. In some embodiments of the invention, the human limbal stem cells used in the systems and methods of the invention are characterized to identify one or more biomarkers. In the working embodiment of the invention that are disclosed herein, prior to being placed in the first cell culture compartment these cells are for example, examined to observe p63α expression levels. In typical embodiments of the invention, the mammalian feeder cells used with the human limbal stem cells comprise human bone marrow derived mesenchymal stem cells, human adipose derived mesenchymal stem cells, or human limbal fibroblasts.

The working embodiments of the invention show that different feeder cells can support limbal stem cell growth using systems incorporating the porous membrane and/or the tissue explant methodology. In specific embodiments of the invention, the different feeder cells include but are not limited to mouse 3T3-J2 cells, human bone marrow-derived mesenchymal stem cells (BM-MSCs), human adipose-derived mesenchymal stem cells (ASCs) and human limbal fibroblasts (LFs). By using the porous membrane, artisans can further reduce the chance of xenogenic contamination due to the separation of the feeder cells from LSCs by the membrane. The systems of the invention provide additional benefits by simplifying the process of changing feeder cells as well as by assuring minimum disturbance on limbal stem cells.

Various types of cells discussed herein can be identified by a number factors including the in vivo locations from which they are obtained. In certain embodiments of the invention, the human limbal stem cells or feeder cells used in the systems and methods of the invention are examined (either before, during or after being placed in the systems disclosed herein) in order to observe cellular morphology and/or to characterize the expression of one or more biomarkers, for example, the expression of, or expression levels of cellular proteins (see, e.g. the Examples below). In this context, the working Examples also include descriptions of cellular morphologies associated with cells discussed herein (e.g. a morphological characteristic of stem cells) as well as common protocols used in the phenotypic analyses of these cells, for example immunohistochemistry and/or qRT-PCR.

Those of skill in this technology understand that cells useful in embodiments of the invention such as limbal stem cells of the corneal epithelium can be characterized by a number of complementary factors such as the in vivo site from which they are obtained, and/or their morphology or size (e.g. average diameter), as well as the presence, absence and/or expression levels of biomarkers such as the ATP-binding cassette subfamily G member 2 (ABCG2), Δp63α, stage-specific embryonic antigen-4 (SSEA4), N-cadherin, and cytokeratins (K) such as K1, K3, K10, K12, K14 or K15, (see, e.g. Nakatsu et al., Investigative Ophthalmology & Visual Science 2011; 52:4734-4741; Truong et al., Invest Ophthalmol Vis Sci. 2011; 52:6315-6320; Dua et al., Surv Ophthalmol. 2000 March-April; 44(5):415-25; Watson et al., Curr Eye Res. 2013 Apr. 10. [Epub ahead of print]; Meyer-Blazejewska et al., Invest Ophthalmol Vis Sci. 2010 February; 51(2):765-74; and Rama et al., N Engl J Med 2010; 363:147-55). In illustrative embodiments of the invention, the human limbal stem cells exhibit an expression profile characterized by examining the expression of one or more of ATP-binding cassette subfamily G member 2 (ABCG2), Δp63α, stage-specific embryonic antigen-4 (SSEA4), N-cadherin, and cytokeratins (K) such as K1, K3, K10, K12, K14 or K15. In embodiments of the invention, other characteristics of the human limbal stem cells or feeder cells are also identified or characterized, for example cellular size or morphology.

All of various types cells discussed herein can be characterized by a number of art accepted methods. Characteristics of mesenchymal stem cells are discussed, for example, in Stockl et al., Cell Physiol Biochem. 2013; 31(4-5):703-17. Characteristics of hair follicle stem cells are discussed, for example, in Ohyama, Dermatology 2007; 214; 342-351. Characteristics of skin epithelial stem cells are discussed, for example, in Tan et al., Development. 2013 April; 140(7):1433-44. Characteristics of human limbal stromal cells or corneal stromal cells are discussed, for example, in Sosnova et al., STEM CELLS, Volume 23, Issue 4, pages 507-515, April 2005 and Polisetty et al., Mol Vis. 2008; 14: 431-442.

Embodiments of the invention include methods for efficiently cultivating LSCs using human feeder cells. In one embodiment, the method comprises using the porous membrane to provide a close proximity between the stem cells and the feeder cells in order to allow for optimal expansion of the LSCs. In this method, primarily isolated LSCs and growth-arrested feeder cells are seeded on the opposite sides of the porous membrane respectively. In an alternative embodiment of the invention, methods for cultivating LSCs using human feeder cell types involves cultivating a small (e.g. 1×2 mm) limbal explant on the porous membrane or on human feeder cells. In one embodiment of the invention, the method for cultivating human LSCs using limbal explant comprises dissecting a 1×2 mm sclerocornea tissue containing ½ stromal tissue. The method further comprises placing feeder cells directly on the porous membrane and adding culture media to the culture.

In some working embodiments of the invention that are disclosed herein, a Millipore cell culture insert is invertedly placed in a large petri dish. One ml of medium containing the feeder cells is then added carefully onto the top of the membrane, which is cultured in the incubator for 4 hours to overnight to allow the feeder cells to attach to the bottom side of the membrane. Then the insert is placed into a well and primarily isolated limbal stem cells or tissue explant are added on top of the insert. The growth medium is changed periodically (e.g. every two days) until cell growth is desired for collection.

Aspects of the invention include systems for culturing human limbal stem cells comprising a container comprising a culture media for human limbal stem cells, a limbal tissue sample explant disposed in the culture media wherein the tissue sample explant comprises human limbal stem cells and this tissue sample has one side that is less than 1 mm and another side that is greater than 1 mm (e.g. 0.5×2 mm); and mammalian feeder cells disposed in the culture media at a location proximal to the human limbal stem cells so that soluble factors produced by the feeder cells migrate to the human limbal stem cells. In some embodiments of the invention, this system includes a porous membrane disposed in the culture media so as to form a first cell culture compartment and a second cell culture compartment, wherein the porous membrane allows soluble factors to migrate between the first cell culture compartment and the second cell culture compartment while simultaneously preventing cells from migrating between the first cell culture compartment and the second cell culture compartment.

Another aspect of the invention is a method of maintaining human limbal stem cells in an undifferentiated phenotype by culturing the human limbal stem cells in the systems disclosed herein. The limbal stem cell phenotype is known in the art and can be characterized by its expression levels of markers including K15, K19, Bmi-1, P63, P63α, ABCG-2, Notch-1, K3/K12 and Desmoglein-3 (see, e.g. Meyer-Blazejewska et al., Invest Ophthalmol Vis Sci. 2010 February; 51(2):765-74). Yet another aspect of the invention is a method of facilitating the proliferation of human limbal stem cells within cell clusters, the method comprising culturing the human limbal stem cells in the systems disclosed herein. Typically in these methods, the human limbal stem cells are disposed in the system at a location and in an orientation selected to influence the polarity of the growing cells.

Additional aspects of the invention include methods of generating cells having a human limbal stem cell phenotype from a related type of stem cell. Typically, such methods include disposing at least one of hair follicle stem cells, skin epithelial stem cells, embyronic stem cells or induced pluripotent stem cells in the a cell culture compartment of the systems disclosed herein. Such methods further include disposing at least one of disposing at least one of human limbal stromal cells or corneal stromal cells in a second cell culture compartment of the systems disclosed herein. In these methods, soluble factors to migrate from the cells in the second cell culture compartment to the first cell culture compartment, wherein the migration of the soluble factors results in the generation of human limbal stem cells from the hair follicle stem cells, epidermal stem cells, embyronic stem cells or induced pluripotent stem cells, so that cell having a human limbal stem cell phenotype are generated. In this context, a “human limbal stem cell phenotype” refers to cellular phenotype that exhibits the functional characteristics that the cells to restore vision in individuals affected with LSCD. In an illustrative embodiment of the invention, skin epithelial stem cells are disposed in the first cell culture compartment and human limbal stromal cells are disposed in the second cell culture compartment and factors produced by the limbal stromal cells modulate the differentiation of the skin epithelial stem cells in a manner that generates human limbal stem cell pheotype.

In embodiments of the invention, both cell sheet and limbal explant method lead to a higher proliferation rate for all four types of human feeder cells tested, compared to the conventional culture methods. The method as described in the embodiments of the present invention can maintain stem cell phenotype of the LSCs, i.e., uniformly small compact undifferntiated epithelial cell morphology and high level of the stem cell marker expression. Additionally, porous membrane systems can help keep a close and even proximity between the limbal stem cells and the feeder cells without physical competition for the growth surface. This allows for a better support of the growth of LSCs. In addition, due to the physical separation of the LSCs and feeder cells, isolation of a pure population of the LSCs is possible.

Embodiments of the invention show that significant amounts of LSCs can be generated using the methods as described above from a 1×2 mm limbal tissue in a potentially xenobiotic-free system. This method enables expansion of autologous LSCs for patients with limbal stem cell deficiency. Those skilled in the art will recognize that embodiments of the present invention can be used to culture any cells whose growth needs the coculture of feeder cells, in particular, stem cells. The material and the pore size of the membrane can be further engineered to have a better control of cell-cell contact and to reduce the chance of contamination from feeder cells. Additionally, explant method can be further optimized to improve the generation of cells having a limbal epithelial cell phenotype.

### EXAMPLES

### Example 1

### Illustrative Methods and Materials Useful with Embodiments of the Invention

### Human Sclerocorneal Tissue

For the working examples disclosed herein, human sclerocorneal tissue was obtained from the Illinois Eye Bank (Watson Gailey, Bloomington, Ill.) and the Lions Eye Institute for Transplant and Research (Tampa, Fla.). Tissue donors ranged in age from 20 to 65 years. Experimentation on human tissue adhered to the tenets of the Declaration of Helsinki. The experimental protocol was evaluated and exempted by the University of California, Los Angeles Institutional Review Boards.

The tissues were preserved in Optisol (Chiron Ophthalmics, Inc., Irvine, Calif.), and the death-to-preservation time was less than 8 hours.

### Preparation of Limbal Epithelial Cell Culture

Limbal epithelial cells were isolated from corneoscleral rims as previously described (see, e.g. Truong et al., Invest Ophthalmol Vis Sci. 52, 6315, 2011). In brief, the residual blood vessels, iris, endothelium, Tenon's capsules, and conjunctiva were removed from the rim. The rim was digested in 2.4 U/ml Dispase II (Roche, Indianapolis, Ind.) in SHEM5 growth medium (DMEM/F12 medium) (Gibco, Grand Island, N.Y.) supplemented with N-2 (Gibco), 2 ng/ml epidermal growth factor (EGF; Gibco), 8.4 ng/ml cholera toxin [Sigma-Aldrich, St. Louis, Mo.), 0.5 μg/ml hydrocortisone (Sigma-Aldrich), 0.5% dimethyl sulfoxide (DMSO; Sigma-Aldrich), 5% fetal bovine serum (FBS, Invitrogen, Carlsbad, Calif.), penicillin/streptomycin (Invitrogen) and gentamicin/amphotericin B (Invitrogen) for 2 hours at 37° C. Sheets of limbal epithelium were scraped from the limbus and pipetted up and down to break the cell sheets into smaller clusters and more evenly distribute the clusters in the medium. Some clusters were further treated with 0.25% trypsin and 1 mM EDTA (Gibco) for 10-15 min at 37° C. to obtain single-cell suspensions. For the in vitro propagation of limbal stem/progenitor cells, epithelial cells, either in cell clusters or in single-cell suspension, were seeded at a density of 300 cells/cm². The cell proliferation rate was evaluated as the total number of epithelial cells recovered from the culture after moving the feeder cells. The absolute number of cells from each culture method was defined as the total number of cells that were produced per limbal epithelial cells seeded.

To prepare tissue explants for culture, corneoscleral rims were separated from residual blood vessels, iris, endothelium, Tenon's capsules, and conjunctiva and then cut into pieces that were approximately 2×2 mm. The explant pieces were placed on the growth surface with the epithelium side facing up. Only one explant piece was cultured per well or per insert.

### Standard and Sandwich Culture Methods

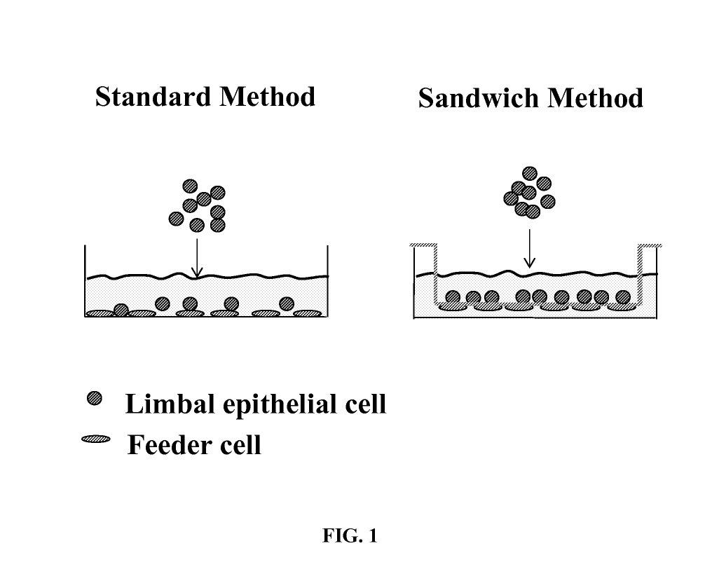

Subconfluent murine 3T3-J2 cells (from Howard Green, Harvard Medical School, Boston, Mass., USA) were treated with 4 μg/ml of mitomycin C (Sigma-Aldrich) for 2 h, and plates were seeded with 3×10⁴ cells/cm². For the standard culture method, suspensions of single limbal epithelial cells, cell clusters, or explants were seeded directly onto the 3T3 feeder cells (FIG. 1A). Standard culture in which single limbal epithelial cells was seeded directly on 3T3 feeder cells was used as control. Alternatively, 6-well inserts with polyethylene terephthalate [PET] membrane with a pore size of 1 μm (Millipore, Billerica, Mass.) were placed upside down into 6-well plates, and 1 ml of medium containing the 3T3 feeder cells was carefully loaded onto the bottom side of each PET membrane (FIG. 1B). The feeder cells were allowed to attach to the membrane at 37° C. The inserts were placed upright into the 6-well plates, and the limbal epithelial cells or explants were seeded onto the inner side of the membrane. The PET membrane with 1 μm pore size was selected because it has been reported that only the 1 μm pore can effectively minimize the migration of mouse embryonic fibroblast feeder cells to zero during culture while the 3 and 8 μm pores cannot (see, e.g. Kim et al., Stem Cells. 25, 2601, 2007). This method of 3D culture is called the “sandwich method” in the rest of the paper. The limbal epithelial cells were cultured for 14-21 days. The medium was replaced every 2-3 days.

### RNA Isolation, Reverse Transcription and Quantitative Real-Time PCR

After culture, epithelial cells were collected, and RNA was extracted (RNeasy Mini Kit, Qiagen, Valencia, Calif.), treated with DNase (DNA-free kit, Ambion, Austin, Tex.), and reverse-transcribed into cDNA (SuperScript II, Invitrogen) according to the manufacturer's instructions. Transcripts were detected by using the Kapa Sybr Fast qPCR kit (Kapa Biosystems, Woburn, Mass.). Cycle conditions were as follows: the reactant was denatured for 20 s at 95° C.; amplified for 40 cycles (temperatures in each cycle were 95° C. for 3 s, 60° C. for 20 s, and 72° C. for 8 s); and subjected to a melting curve program to obtain the dissociation curve. The primers used in quantitative real-time PCR (qRT-PCR) were listed in Table 1 below.

TABLE 1
Primers Used in qRT-PCR

| | Forward primer | Reverse primer |
|---|---|---|
| Marker | (5′-3′) | (5′-3′) |
| ABCG2 | AACCTGGTCTCAACGCCATC | GTCGCGGTGCTCCATTTATC |
| | (SEQ ID NO: 1) | (SEQ ID NO: 2) |
| ΔNp63 | TCCATGGATGATCTGGCAAGT | GCCCTTCCAGATCGCATGT |
| | (SEQ ID NO: 3) | (SEQ ID NO: 4) |
| N-cad | AGCCAACCTTAACTGAGGAGT | GGCAAGTTGATTGGAGGGATG |
| | (SEQ ID NO: 5) | (SEQ ID NO: 6) |
| K14 | GACCATTGAGGACCTGAGGA | ATTGATGTCGGCTTCCACAC |
| | (SEQ ID NO: 7) | (SEQ ID NO: 8) |
| K12 | CCAGGTGAGGTCAGCGTAGAA | CCTCCAGGTTGCTGATGAGC |
| | (SEQ ID NO: 9) | (SEQ ID NO: 10) |
| Ki67 | CTTTGGGTGCGACTTGACG | GTCGACCCCGCTCCTTTT |
| | (SEQ ID NO: 11) | (SEQ ID NO: 12) |
| GAPDH | CGACCACTTTGTCAAGCTCA | AGGGGTCTACATGGCAACTG |
| | (SEQ ID NO: 13) | (SEQ ID NO: 14) |

### Immunocytochemistry and Quantitation

Expanded epithelial cells were cytospun on slides by a cytocentrifuge (Cytofuge; Fisher Scientific, Hampton, N.H.) and stored at −20° C. until use. Cytospin slides were fixed with 4% paraformaldehyde at room temperature for 10 min and washed 3 times with phosphate-buffered saline (PBS) containing 0.3% Triton X-100 (Sigma-Aldrich). PBS with 10% donkey serum was used for 30 min at room temperature to block the sections. Sections were incubated with one or more primary antibodies diluted in PBS with 1% bovine serum albumin (BSA) overnight at 4° C. in a moisture chamber. Sections were washed 3 times with PBS with 1% BSA, incubated with one or more secondary antibodies diluted in PBS with 1% BSA at room temperature for 1 h, and washed with PBS containing 1% BSA and 0.3% Triton X-100. Nuclei were labeled with Hoechst 33342 (4 μg/ml; Invitrogen) at room temperature for 15 min, washed 5 times with PBS, and mounted in Fluoromount medium (Sigma). The primary and secondary antibodies and their dilution ratios are listed in Table 2 below.

TABLE 2
Primary Antibodies Used in Immunocytochemistry
Images were taken by a confocal microscope (Confocal Laser Scanning
Microscopy; Olympus, San Jose, CA) and an image capture system
(Fluoview FV10-ASW 3.1 Viewer; Olympus). The nuclear intensity of
p63α was determined by the Definiens Tissue Studio software
(Larchmont, NY).

| Marker | Catalog No. | Company | Dilution |
|---|---|---|---|
| P63α | 4892S | Cell Signaling | 1:100 |
| K14 | K14 Ab (Clone LL002) | NeoMarkers/Fisher | 1:2 |
| | | Scientific | |
| K12 | Sc-25722 | Santa Cruz Biotechnology | 1:100 |

### Statistical Analysis

Student's t-test was performed to analyze the data. Each error bar represents the standard error of the mean (SEM) from at least 3 experiments. P values <0.05 were considered to indicate statistical significance.

### Example 2

### Illustrative LSC Cultures

LSC Cultures Generated from Single-Cell Suspensions

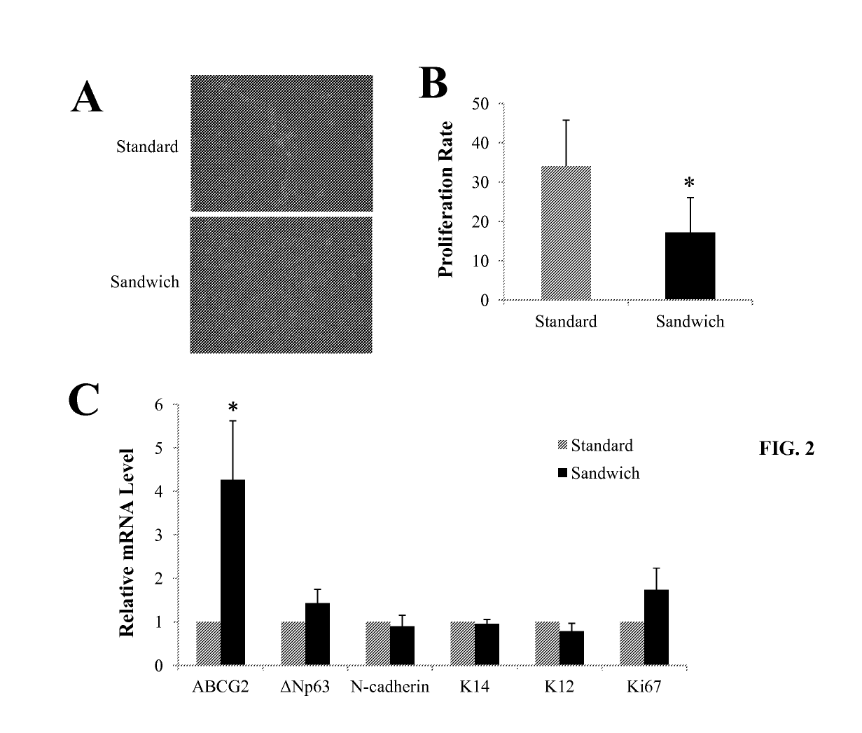

LSCs generated from single-cell suspension cultured by the standard or sandwich method showed similar compact, cuboidal epithelial morphology (FIG. 2A). LSCs cultured by using the sandwich method had better stem cell phenotypes than did those cultured by using the standard method: the expression of ABCG2 was 2.8-fold greater in LSCs grown with the sandwich method (p<0.05), and the expression levels of other markers were comparable between LSCs obtained by either method (FIG. 2C).

LSC Cultures Generated from Cell Clusters

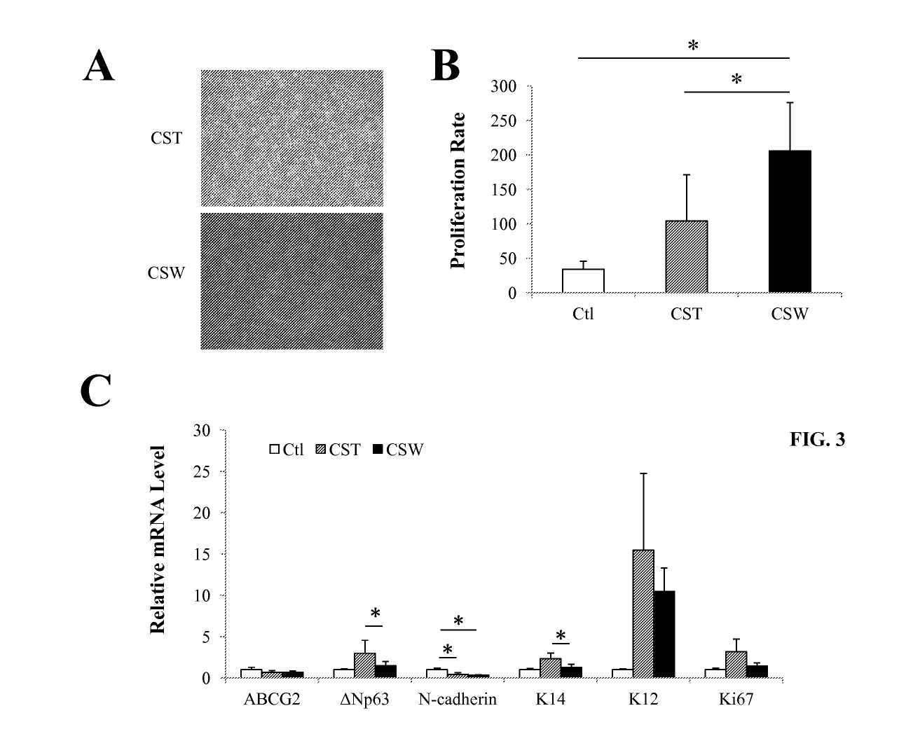

Cells derived from clusters cultures using standard (cluster standard method) or sandwich methods (cluster sandwich method) were compact and displayed a cuboidal, epithelial stem-cell morphology (FIG. 3A). The proliferation rates of cells obtained from cell clusters in the standard and sandwich cultures were greater than that of the control (i.e., a single-cell suspension cultured directly on feeder cells). The control was amplified 34-fold, whereas the clusters grown in standard culture and sandwich culture were amplified 104-fold and 206-fold, respectively (FIG. 3B).

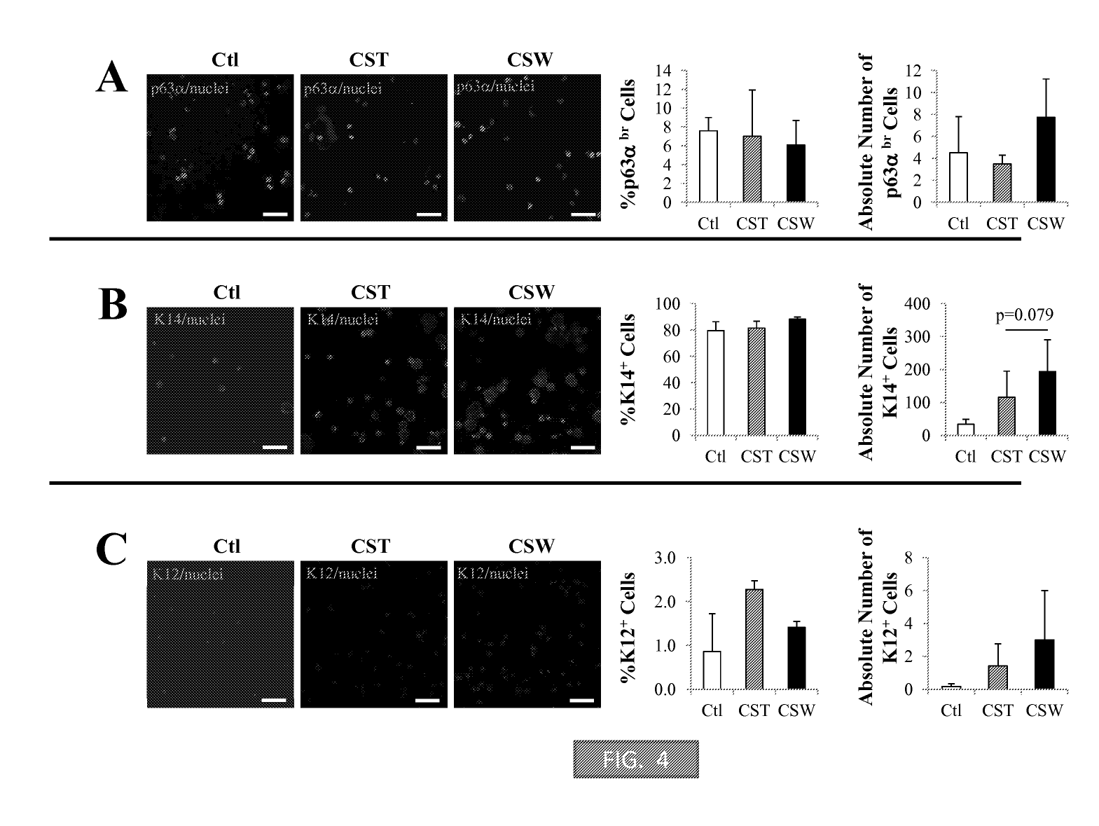

Limbal epithelial cells from the control culture expressed a higher level of N-cadherin mRNA than did cells derived from clusters standard culture (1.3-fold higher) and cluster sandwich culture (1.8-fold higher; p<0.05 for both comparisons). Lower mRNA levels of putative LSC markers, ΔNp63 and K14 were seen in cells derived from clusters sandwich method and cluster standard method (lower by 34% and 43%, respectively; p<0.05 both comparisons with the control) (FIG. 3C). The percentage of p63α-bright (p63αbr) cells in the cultured LSCs is a prognostic factor of clinical success after transplantation in humans (see, e.g. Rama et al., The New England journal of medicine. 363, 147, 2010). We then investigated the portion of p63αbr cells in our cultures. Immunocytochemistry data showed no significant differences in the percentages of p63αbr cells among the control, cluster standard method and cluster sandwich methods (FIG. 4A). The percentage of K14+ cells was also evaluated (FIG. 4B). All three types of cultures contained 6-7% p63αbr cells and 80-90% K14+ cells. Cluster sandwich method produced slightly higher absolute numbers of p63αbr cells and K14+ cells than did the control and the standard method; however, the differences did not reach significance. The percentages and absolute numbers of K12+ cells were extremely low in all 3 groups (0.8-2.3% and 0.1-3, respectively) (FIG. 4C).

LSCs Generated from Limbal Explant Cultures

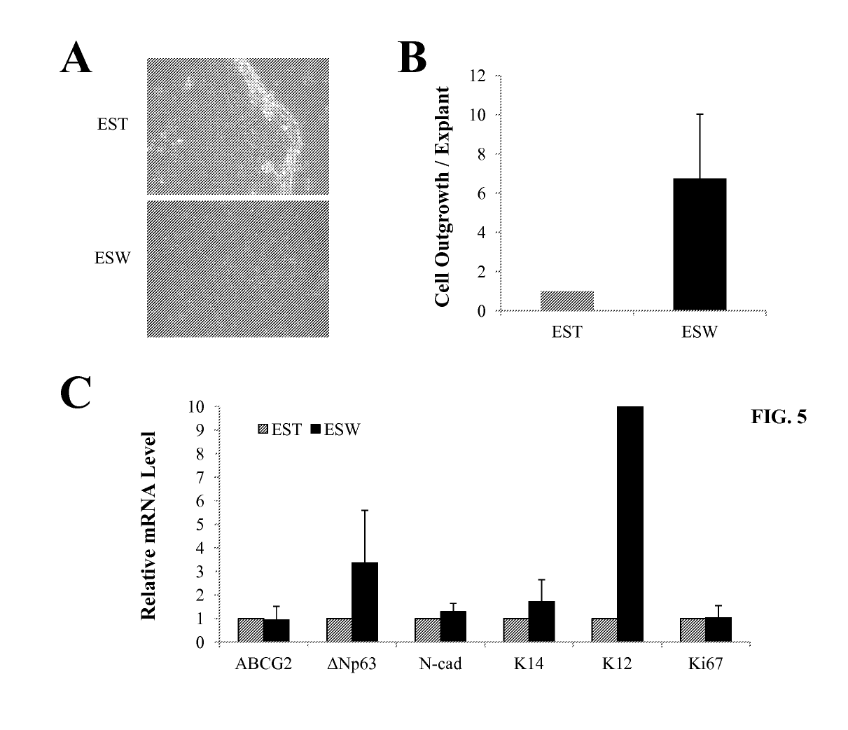

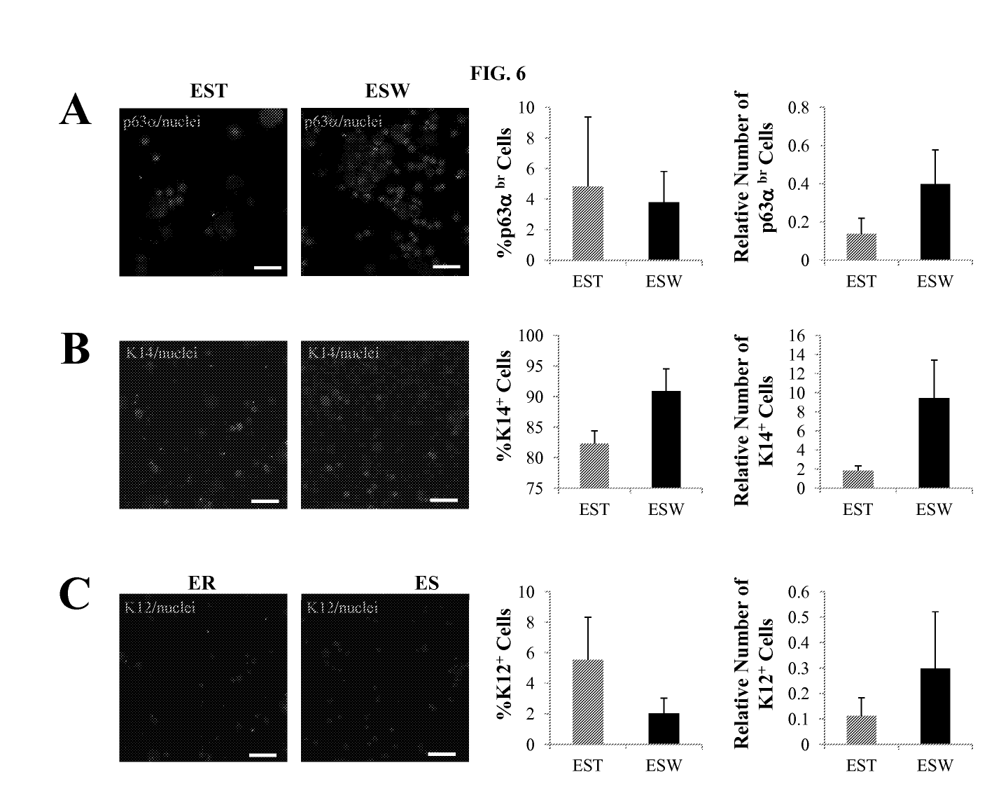

The 2×2 mm explants were seeded either on top of the feeder cells in a cell-culture petri dish (standard method) or on top of a porous membrane with feeder cells seeded on the bottom side of the membrane (sandwich method). Both methods produced cells with small compact cuboidal undifferentiated epithelial morphology (FIG. 5A). The proliferation rate of explant outgrowth in the sandwich culture was 6-fold greater than that in the standard culture (FIG. 5B); however, the difference did not reach significance. Explants in the sandwich culture tended to have higher levels of ΔNp63, K14, and K12 mRNA than did explants in the standard method, but none of the differences reached significance (FIG. 5C). Immunocytochemical analysis showed that in comparison with explants in the standard culture, explants in the sandwich culture generated a comparable percentage of p63αbr cells, a higher percentage of K14+ cells, and a lower percentage of K12+ cells (FIGS. 6A, 6B, and 6C). However, the differences failed to reach significance. Both samples contained a low percentage of p63αbr cells (3-5%), a high percentage of K14+ cells (80-90%), and a low percentage of K12+ cells (1.5-5.5%). The absolute numbers of p63αbr cells, K14+ cells, and K12+ cells in the sandwich culture were comparable to those generated in the standard culture.

### Discussion of Experimental Data

Compared with the standard 2D culture method, the 3D sandwich method disclosed herein better resembles the in vivo environment of LSCs. Our results showed that no matter which form of LSCs (i.e., single-cell suspension, cell cluster, or explant) was seeded, cells derived from the sandwich culture method had phenotypes comparable to those of stem cells. Moreover, the expansion rates of epithelial cells from the cluster and explant sandwich method were greater than those from the cluster and explant standard method, respectively.

Without being bound by a specific scientific theory or mechanism of action, we propose several hypotheses to explain the increased proliferation rate and the concurrent maintenance of the stem cell phenotypes in the sandwich culture sytems disclosed herein. First, the evenly close proximity of all epithelial cells to feeder cells in the sandwich culture may have provide sufficient growth support to promoted a better proliferation rate while maintaining the less differentiated stem/progenitor phenotype. Previous reports indicated that at the edge of LSC colonies exhibited more stemness and more active proliferation than the cells in the center of the colonies (see, e.g. Secker, G. A., and Daniels, J. T. Limbal epithelial stem cells of the cornea. 2008; and Higa et al., Invest Ophthalmol Vis Sci. 50, 4640, 2009); these results provide evidence that close proximity to the feeder cells support the stem cell phenotype of LSCs by providing adequate nutrient levels and/or cell-cell contact. Second, the sandwich method may help to maintain the polarity of LSCs and thus the proliferation and maintenance of phenotype. Limbal epithelial cells, as a type of nonkeratinized stratified epithelium, have an apical-basolateral polarity, which is crucial for their proliferation, differentiation, and proper functioning (see, e.g. St Johnston et al., Cell. 141, 757, 2010; and Martin-Belmonte et al., Nat Rev Cancer. 12, 23, 2012). In the standard method, feeder cells are seeded to the side of the epithelial cells, whereas in the sandwich method, feeder cells are located below the epithelial sheet. This arrangement in the sandwich culture may better maintain stem cell polarity and thus may better maintain stemness and sustain proliferation.

It is interesting that the growth rate of cells derived from single-cell sandwich method was lower than that of cells derived from single-cell standard method. In some instances no growth from single-cell suspensions in sandwich culture was observed. It is possible that for survival and proliferation, single LSC may need direct contact with feeder cells and that the PET membrane in the sandwich culture may not permit sufficient contact. Another possibility is that the PET membrane is not an ideal substrate for the initial attachment of single limbal epithelial cells for proliferation.

Lee and colleagues described a similar 3D culture method (see, e.g. Kim et al., Stem Cells. 25, 2601, 2007; and Hwang et al., Biomaterials. 31, 8012, 2010). In their method of culture, human embryonic stem cells (hESCs) were placed on top of a porous PET membrane with feeder cells attached to the other side. This 3D method effectively propagated hESCs and maintained their stem cell phenotype. There is still cell-cell contact via the pores between the feeder cells and the hESCs, and maintaining the physical cell-cell contact might be important in supporting the expansion of hESCs. However, no comparison was made between this 3D method and the conventional method of culture for hESCs. Their reports focused on the benefit that this 3D method can effectively separate stem cells and feeder cells and thus can facilitate stem cell harvest and reduce xenogenic contamination by the feeder cells. This benefit also applies to our sandwich method. In the standard method of culture, limbal stem/progenitor cells can be harvested by removing the feeder cells through enzymatic or chemical dissociation. Removal of feeder cells can be incomplete, and the dissociation process can be harmful to the cultured stem/progenitor cells, which tend to locate at the edge of colonies. In the sandwich method, LSCs are separated from feeder cells by the membrane; therefore, the cultured LSCs can be easily removed by enzymatic digestion within the insert. Cell migration will not occur with a pore size less than 3.0 μm. Therefore, contamination of feeder cells in the cultured LSCs is unlikely.

In summary, a novel 3D sandwich method described in the current study can efficiently expand LSCs without contamination by feeder cells.

### Example 3

### Transdiffentiation of Human Limbal Stem Cells

The corneal epithelium is constantly renewed and maintained by the corneal epithelial stem cells, or limbal stem cells (LSCs) that are presumed to reside at the limbus, the junction between the cornea and conjunctiva. When the LSCs are deficient and unable to repopulate the corneal surface, the cornea surface can become opaque. Limbal stem cell deficiency (LSCD) has been recognized as one of the causes of significant visual loss and blindness (see, e.g. Dua et al., Indian J Ophthalmol 2000; 48:83-92; and Grueterich et al., Surv Ophthalmol 2003; 48:631-646.).

In patients who have LSCD restricted to one eye, a small biopsy can be obtained from the healthy eye and autologous LSCs from this biopsy can be expanded ex vivo using mouse 3T3 cells as feeder cells (see, e.g. Rama et al., N Engl J Med 2010; 363:147-155). Transplantation of these autologous LSCs onto the diseased eye has successfully reconstructed a transparent ocular surface. To eliminate the risk of cross contamination from animal products, a xenobiotic-free culture system to efficiently expand LSCs has been developed. In patients who have bilateral disease, autologous oral mucosal epithelial cells can be successfully expanded in culture and transplanted onto the ocular surface (see, e.g. Nishida et al., N Engl J Med 2004; 351:1187-119). However, the oral mucosal epithelial cells do not transdifferentiate into the corneal phenotype and the postoperative visual outcome is still poor (see, e.g. Satake et al., Ophthalmology 2011; 118:1524-1530; Madhira et al., Molecular vision 2008; 14:189-196; and Nakamura et al., The British journal of ophthalmology 2011; 95:942-946). In this context, additional embodiments of the invention relate to overcoming these problems via methods that regenerate functional LSCs from human skin epithelial stem cells, LSCs that can be used for transplantation to patients with bilateral LSCD.

Human skin epithelial stem cells and corneal epithelia develop from surface ectoderm and thus are closely related, despite the mature skin epithelial cells and corneal epithelial cells having very distinct phenotypes. Successful generation of corneal epithelial-like cells from reprogramming of mouse hair follicle stem cells in vitro without genetic modification and restoration of a corneal surface in LSCD mice using these induced corneal epithelial cells provides that lineage reprogramming of skin epithelial stem cells into a functional corneal lineage is feasible with human cells (see, e.g. Meyer-Blazejewska et al., Stem Cells 2011; 29:57-66). Lineage reprogramming can provide an abundant and easily accessible autologous source of donor cells for ocular surface reconstruction in bilateral LSCD. Another major advantage of this direct reprogramming approach over using induced pluripotent stem cells is that tumorigenic risk is minimal because no genomic modification of the induced corneal epithelial cells.

Undifferentiated stem/progenitor cells have the plasticity to differentiate and transdifferentiate. There are two locations where the SECSs are located, the interfollicular epidermis and the hair follicle and epidermal stem cells can be obtained from both locations. Once human SESCs are isolated and cultured, we can induce transdifferentiation of SESCs to the corneal lineage using a co-culturing method in the 3-dimentional culture systems disclosed herein.

SESCs and LSCs share very similar regulatory mechanisms. For example, both Wnt and Notch signaling regulate the lineage determination between the SESCs and LSCs (see, e.g. Mukhopadhyay et al., Development 2006; 133:2149-2154; and Vauclair et al., Dev Cell 2007; 13:242-253). In this context, modulators of Wnt and Notch signaling, respectively, can be used to further induce or increase the efficiency of transdifferentiation.

TABLE 3
Upregulated genes in human HF stem cells and LSC niche
Upregulated genes in human HF stem cells and LSC niche

| HF stem cells | LSC niche |
|---|---|
| Trinucleotide repeat containing 9 (TNRC9) | − |
| DIO2 | + |
| PHLDA1 | + |
| ANGPTL2 | ANGPTL1 |
| WIF1 | + |
| Regulated in glioma (RIG) | − |
| Dikkopf homolog 3 (Xenopus laevis) (DKK3) | Dkk2 |
| Calcium/calmodulin-dependent protein kinase II (CaMK2A) | CaMK2D |
| Dihydropyrimidinase-like 2 (DPYSL2) | + |
| GPM6B | + |
| Dihydropyrimidinase-like 3 (DPYSL3) | + |
| Decorin (DCN) | − |
| Serine (or cysteine) proteinase inhibitor, clade F, member 1 | + |
| (SERPINF1) | |
| Ca2+ - dependent activator protein for secretion 2 | − |
| (CADPS2) | |
| Transforming growth factor, beta 2 (TGFB2) | TGFBR2 |
| KRT15 | + |
| Frizzled homolog 1 (Drosophila) (FZD1) | + |
| Nuclear factor of activated T cells, cytoplasmic, | + |
| calcineurin-dependent 1 (NFATC1) | |
| FST | + |
| Calmodulin 1 (phosphorylase kinase, delta) (CALM1) | − |
| Dopachrome tautomerase (dopachrome delta-isomerase, | + |
| tyrosine-related protein 2) (DCT) | |
| TOX high mobility group box family member 3 (TOX3) | + |

### Human SESCs and LSCs are Closely Related.

For patients with bilateral LSCD, corneal epithelial stem cells must be regenerated from other sources than the cornea. Successful generation of induced-pluripotent stem cells using genetic reprogramming opens up new avenues for patient-specific stem cell therapy (see, e.g. Takahashi et al., Cell 2006; 126:663-676; and Lowry et al., Proc Natl Acad Sci USA 2008; 105:2883-2888). However, the clinical application of this very technology is limited due to the risk of malignant transformation as well as the low efficiency of reprogramming and directed differentiation to obtain the desired cell type (see, e.g. Okita et al., Nature 2007; 448:313-317). In this regard, skin epithelial stem cells (SESCs), cells which share the same developmental lineage as LSCs and are easily accessible from the same patient in large quantity, represent an excellent alternative cell source.

Microarray data studies of the human LSCs niche have lead to our discovery that both SESCs and LSCs (both of which develop from ectoderm) share strikingly similar molecular markers based on the comparison of the published skin hair follicle (HF) bulge stem cells markers (see, e.g. Ohyama et al., J Dermatol Sci 2007; 45:147-150; and Ohyama et al., J Clin Invest 2006; 116:249-260). Comparative gene profiling was performed on the human limbus, where the LSCs are located against cornea, which is lined with mature corneal epithelial cells. These studies elucidate the unique molecular markers and regulatory components of LSCs and their niche. These markers include K14, K15, Np63, Wnt inhibitory factor 1, frizzled 1, and nuclear factor of activated T-cells cytoplasmic 1 (NFATC1). Among the top 21 genes that are upregulated in the human HF bulge stem cells compared to the non-bulge region, 13 of them are also upregulated in the LSCs niche compared to the central cornea (Table 3). In addition, LSC niche expresses four genes in the same family of the genes that are upregulated in the HF stem cells. For example, Dickkopf (Dkk3) and TGFB2 are upregulated in HF bulge stem cells whereas Dkk2 and TGFB2 receptor are upregulated in the LSC niche. Many of these genes are key regulatory factors in the proliferation and differentiation of SESCs. This data provides evidence that SECSs and LSCs are very closely related and share similar phenotype and regulatory mechanisms.

SESCs can be Reprogrammed into Functional Corneal Epithelial Cells.

Stem cells in the HF bulge region can be induced to give rise to neurons, smooth muscle, epidermal epithelium, and melanocytes under appropriate environment. These observations provide evidence that the HF bulge is a unique area harboring pluripotent adult stem cells. In this context, clinical and experimental data indicate that corneal epithelial cells can transdifferentiate into the epidermal phenotype under appropriate conditions, and vice versa. For example, in chronic severe dry eye and Vitamin A deficiency the cornea becomes keratinized and skin-like (see, e.g. Beitch et al., Invest Ophthalmol 1970; 9:827-843; and Vauclair et al., Dev Cell 2007; 13:242-253). Expression of the skin cytokeratin K10 is detected in the transdifferentiated corneal epithelial cells. In another study, corneal basal epithelial cells were dedifferentiated and then reprogrammed to form interfollicular epidermis and HF (see, e.g. Pearton et al., Int J Dev Biol 2004; 48:197-201; Pearton et al., Proc Natl Acad Sci USA 2005; 102:3714-3719).

Murine and rat HF stem cells can be transdifferentiated into corneal epithelial-like cells in conditioned media derived from limbal fibroblasts or limbal tissue extracts, respectively (see, e.g. Blazejewska et al., Stem Cells 2009; 27:642-652; and Yang et al., Cell Biol Int 2009). Limbal fibroblasts provide the LSC niche factors that can induce the transdifferentiation. Upon transplantation of the murine transdifferentiated corneal epithelial cells onto the mouse ocular surface, they can reconstruct the ocular surface with K12 expressing corneal epithelial cells in 80% of the LSCD mice. The corneal epithelial stem cell pool was repopulated, and conjunctival ingrowth, the hallmark of LSCD, was suppressed (see, e.g. Meyer-Blazejewska et al., Stem Cells 2011; 29:57-66). These findings provide evidence that LSC microenvironment can modulate stem cell fate in otherwise committed adult skin stem/progenitor cell population. These rodent studies provide evidence that human SESCs can similarly be induced to transdifferentiate into functional LSCs.

### Regulatory Signaling in Epithelial Fate Determination.

Wingless (Wnt) and Notch signaling play important roles in epidermal development and in fate determination between the corneal and epidermal epithelia. Expression of the Wnt inhibitor Dkk2 in corneal epithelia is maintained throughout development in mice and is upregulated in the human adult limbal region where LSCs are located compared to the central cornea (see, e.g. Ang et al., Gene Expr Patterns 2004; 4:289-295). The corneal surface in Dkk2 null mice takes on a skin-like phenotype two weeks after birth (see, e.g. Mukhopadhyay et al., Development 2006; 133:2149-2154). Adult Dkk2−/− mice show normal skin and pelage development. The ocular surface of these mice has hair growth, sebaceous glands, an absence of the corneal specific marker K12, but expression of K1, indicating an epidermal stratified epithelial phenotype. Wnt/β-catenin activity is also increased in the epithelia of the Dkk2−/− mice.

Interestingly, during the transdifferentiation of rat HF stem cells to corneal-like epithelial cells, β-catenin is down regulated (see, e.g. Yang et al., Cell Biol Int 2009). In contrast, when corneal epithelial cells are reprogrammed into the dermal epithelial lineage, expression of β-catenin and Lef-1 is increased (see, e.g. Pearton et al., Proc Natl Acad Sci USA 2005; 102:3714-3719). Ectopic expression of Wnt inhibitor Dkk1 abolishes the development of HF in mice and β-catenin controls the morphogenesis of HF and differentiation of skin stem cells (see, e.g. Andl et al., Dev Cell 2002; 2:643-653; and Huelsken et al., Cell 2001; 105:533-545). These findings provide evidence that activation of Wnt/β-catenin signaling is required in the formation of skin dermis and Dkk2 is required to down regulate Wnt/β-catenin signaling during corneal development. Interestingly, Dkk1 and Dkk2 are preferentially expressed in human LSC niche (Table 3).

Notch signaling regulates the proliferation and differentiation of epidermal and corneal epithelial cells. In the inducible skin-specific Notch1−/− mice (K5 promoter), epidermal hyperplasia and extensive hyperplasia and keratinization of the corneal epithelia occur (see, e.g. Nicolas et al., Nat Genet 2003; 33:416-421). The change to a dermal phenotype is confirmed by the epidermal epithelial morphology and expression of the epidermal epithelial marker K1.11 The ablation of Notch1 activity leads to activation of the Wnt/β-catenin pathway. This is consistent with increased Wnt/β-catenin activity leading to the epidermal cell fate. Further investigation by Vauclair et al. reveals that Notch1 is not required for corneal development during the embryogenesis. Instead, Notch1 is required to maintain the corneal fate and differentiation into mature corneal epithelial cells in mice (see Vauclair et al., Dev Cell 2007; 13:242-253). Their finding is consistent with the study by Nakamura et al. which shows that Hesl, a downstream gene of Notch signaling is expressed mainly in the LSC compartment and is required to maintain the undifferentiated state of LSCs in mice (see Nakamura et al., Stem Cells 2008; 26:1265-1274).

### Isolation and Growth of Human LSCs and SESCs.

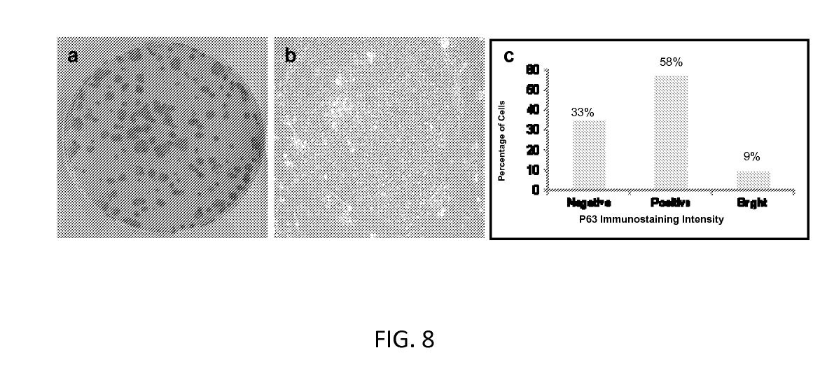

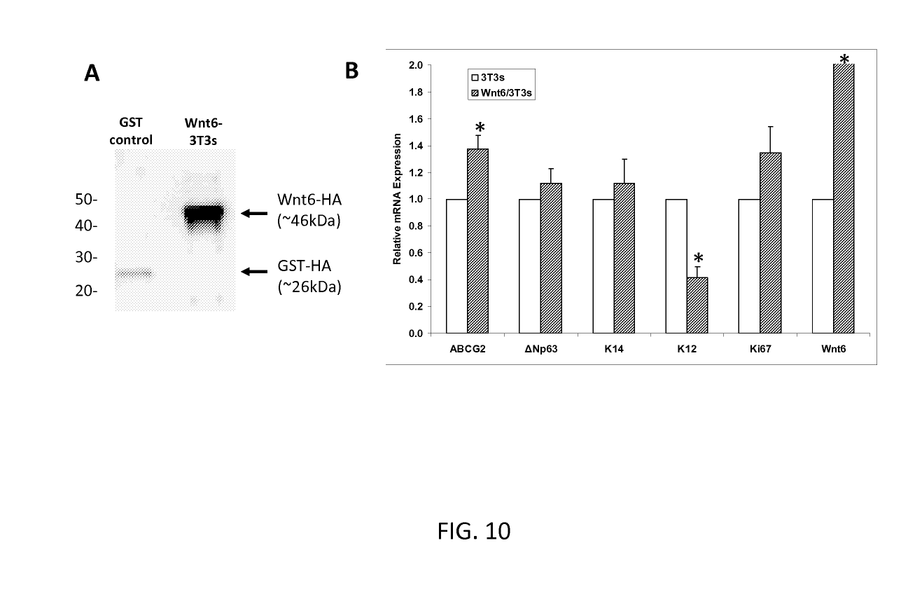

We disclose methods and systems designed to culture and characterize primary human LSCs isolated from sclerocorneal tissues. For example, these limbal epithelial cells can be cultured on NIH 3T3 feeder layers that have been pretreated with mitomycin C to achieve mitotic arrest (FIGS. 8A & 8B). The stem/progenitor phenotype can be characterized by their colony forming efficiency, expression of putative LSCs markers, ATP-binding cassette transporter subfamily G member 2 (ABCG2), ΔNp63α, K14 and low expression level of the differentiation marker K12 by qRT-PCR (FIG. 10) (see, e.g. Nakatsu et al., Investigative Ophthalmology & Visual Science 2011; 52:4734-4741). Our culture system consistently produces on an average of 9% (8-10%) cultured epithelial cells expressing a high level of the stem cell marker p63 (p63− bright cells, FIG. 8C). This efficiency is the same as that reported in the published literature (see, e.g. Di Iorio et al., Microsc Res Tech 2006; 69:983-991). To increase the growth efficiency, we have developed a new 3-dimensional (D) co-culture methods that maximize the contact with the feeder cells.

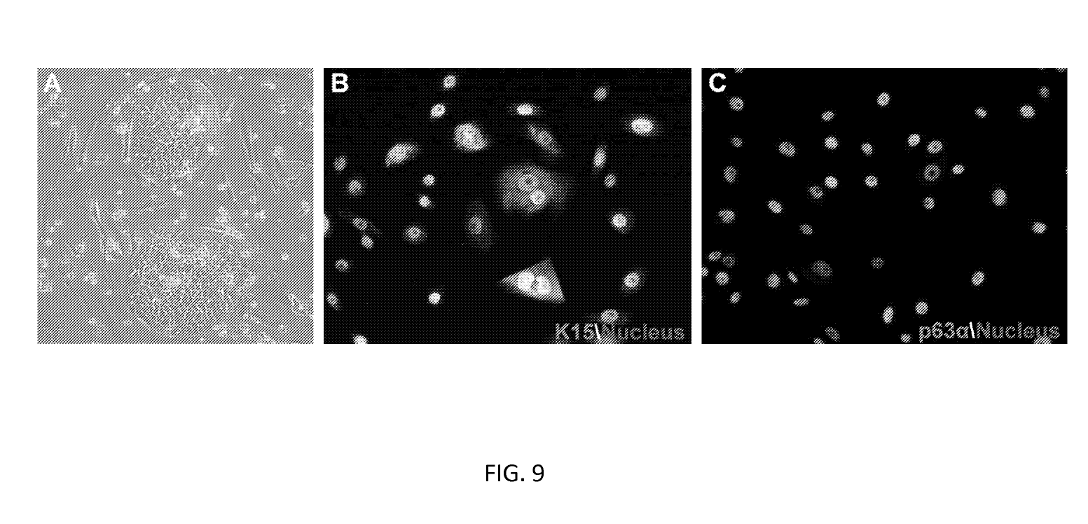

A number of protocols have been established to isolate human skin epidermal stem/progenitor cells. There are two main epidermal stem cell compartments in skin: one is at the basal layer of the interfollicular epidermis (IFE) and the other is the bulge region in the HF. In some protocols, we isolate cells directly from the HF that form colonies on mouse 3T3 feeder layers, indicative of a stem/progenitor phenotype (FIG. 9A). In other protocols we isolate epithelial cells from the IFE and the stem cell population was enriched in low Ca2+KSFM (<0.06 mM) using established method (see, e.g. Lorenz et al., Cells Tissues Organs 2009; 189:382-390). These keratinocytes express high levels of K15 and p63α after two passages, indicative of a stem/progenitor phenotype (FIGS. 9B & 9C).

### Wnt and Notch Signaling in the Regulation of LSCs.

qRT-PCR array analysis of Wnt signaling components in the human limbus as compared to the cornea reveals that Wnt2, Wnt6, Wnt11, and Wnt16b are preferentially expressed (>2-fold) in the limbus. This observation provides evidence that Wnt signaling is important in the regulation of LSCs proliferation and differentiation.

NIH 3T3 cells express high level of Wnt2 and Wnt11 but little Wnt6 and Wnt16b. When Wnt6 was expressed in 3T3 feeder cells (FIG. 10), expression of the putative LSC stem cell markers, ABCG2, K14, and ΔNp63α was maintained in co-cultivated primary human LSCs, while expression of K12 was significantly lower (FIG. 10A). The colony-forming efficiency was at the level similar to that seen with LSCs grown on control 3T3 feeder cells. These observations provide evidence that Wnt6 is important in maintaining undifferentiated LSCs in culture.

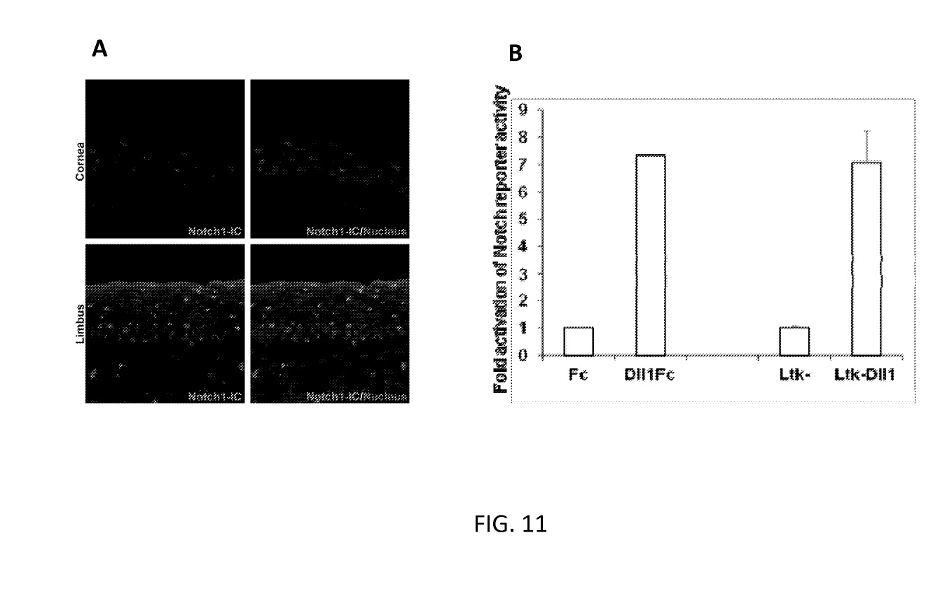

Notch signaling appears to play a pivotal role in the differentiation of human corneal epithelial cells (see, e.g. Ma et al., Invest Ophthalmol Vis Sci 2007; 48:3576-3585). Microarray data shows that Notch1 has a higher expression level in the limbal region compared to the cornea. Only Delta-like (DII) 1 and Jagged (Jag) 1 are expressed in human limbal and corneal epithelium. Immunostaining of the sclerocorneal tissue with an antibody specific for activated Notch1 identified clusters of positive cells in the limbal basal layer as well as in the suprabasal layer (FIG. 11A), provides evidence that Notch signaling is activated in these regions where LSCs reside.

Detection of Notch signaling in the limbal basal layer that harbors the putative LSCs is extremely exciting as it provides evidence that Notch signaling may regulate the growth and differentiation of these progenitors. Notch-expressing cells grown on plates coated with the Notch ligand DII1 activate signaling similar to that induced by co-cultured Dll1-expressing Ltk-cells (FIG. 11B). This simplified method can be used to investigate the function of Notch1 in the reprogramming of SESCS into LSCs and may be applied to the preparation of LSC for clinical applications. Taken together, these findings provide evidence that Wnt and Notch pathways not only play critical roles in the fate determination decision between the epidermal and corneal lineages, but also regulate LSC proliferation and differentiation. Therefore, the appropriate modulation of these two signaling pathways should facilitate the reprogramming of SESCs to LSCs and maintain the undifferentiated state of LSC in culture for expansion prior to transplantation.

### Illustrative Protocols Useful in Aspects of the Invention

### Optimize the Isolation of Human SESCs

A first step in many aspects of the invention disclosed herein is to obtain human SESCs. The less differentiated cells presumably have higher plasticity and therefore have the higher potential to reprogram into LSC. Unlike the stem cells from the HF bulge region which have the potential to give rise to neurons, HF and epidermal epithelium, the stem cells from the IFE appear to commit mostly to epithelial fate. These IFE epidermal stem cells appear to have less potential to reprogram into LSC.

We are able to isolate HF stem/progenitor cells using the clonogenic assay on 3T3 feeder cells and can enrich IFE progenitor cells in a low Ca2+KSFM medium after serial passages (FIG. 9). We can optimize the yield of the stem/progenitor cell population during the initial isolation step. The rationale is that if enough stem/progenitor cells are obtained, their expansion in culture might not be necessary prior to reprogramming; instead, they can be directly used in the co-culture as described below. One can use putative skin epithelial stem cell markers, integrin-β1hi/integrin-α6hi/CD71lo to enrich the stem/progenitor cell population by cell sorting using established protocols (see, e.g. Jiang et al., Histochem Cell Biol 133:455-465; Webb et al., Differentiation 2004; 72:387-395; Li et al., Mol Biol 2005; 289:87-96; Pikula et al., Cell Biol Int 2010; 34:911-915). Both IFE and HF stem cells express high levels of integrin-β1 and integrin-α6 but low levels of CD71.

Briefly in such protocols, fresh human skin tissues (discarded from elective surgery or from National Disease Research Interchange) are incubated with dispase overnight and the epithelial cell sheets are separated from the dermis. Single basal IFE epithelial cells are harvested by trypsin digestion and labeled with anti-integrin-β1, anti-integrin-α6 and anti-CD71 antibodies. The integrin-β1hi/integrin-α6hi/CD71lo population is isolated by flow cytometry. HF bulge stem cells can be isolated using an established protocol (see, e.g. Ohyama et al., J Clin Invest 2006; 116:249-260). Individual follicles are pushed out from the scalp tissues after dispase digestion. The dermal papilla that supports HF development is removed. Single HF cells are obtained by trypsin digestion and the stem cell population, which is CD200hi can be sorted out by flow cytometry. The yield of the stem cell population can be confirmed using colony forming efficiency (CFE) assay on 3T3 cells. Phenotypic analysis of the sorted skin epithelial stem cells will involve immunohistochemistry and qRT-PCR to determine K10, K12, K15, ABCG2 and Np63α expression. The desired stem cell population should be K1/10−, K3/12−, and K14+.

In the event that cell loss/death is excessive during flow cytometry isolation, magnetic bead cell sorting can be used instead of flow cytometry. We have good experience with magnetic bead cell sorting to enrich LSCs and expect that it may be less damaging to the cells (see, e.g. Truong et al., Investigative ophthalmology & visual science 2011; 52:6315-6320). If sufficient numbers of skin epithelia stem cells cannot be obtained after cell sorting, the isolated cells can be expanded in serum-free KSFM medium containing low Ca2+ for 1-2 passages according to established methods (FIG. 9) (see, e.g. Lorenz et al., Cells Tissues Organs 2009; 189:382-390). The minimal number of SESCs is determined by the number of SESCs necessary to obtain a 15-mm confluent cell sheet after co-culture with limbal stromal cells discussed in the section immediately below.

Reprogram SESCs into Corneal Epithelial Cells Using Co-Culture Method

Reprogramming of mouse skin epithelial stem cells into corneal like-epithelial cells has been recently been shown in mice (see, e.g. Blazejewska et al., Stem Cells 2009; 27:642-652). This study indicates that limbal stromal cell conditioned medium has strong influence on the transdifferentiation process, likely due to the soluble factors in the conditioned medium. One can approach to provide the appropriate microenvironment for reprogramming human SESCs. Limbal stromal cells likely provide a better microenvironment than conditioned medium for reprogramming because such co-culture methods provide cell-cell contact that might exert necessary signals towards the corneal fate (in addition to providing the soluble factors of the conditioned medium). Our laboratory has confirmed that we can isolate and culture primary human limbal stromal cells in serial passages.

We have discovered that LSCs do not grow well when they are seeded directly onto the limbal stromal cells. As noted above, we have developed a new 3-dimensional (D) culture system that allows for separation of the feeder cells from the cultured stem cells while providing maximal cell-cell contact between them. Limbal stromal cells support the growth of LSCs using the 3-D method at a comparable efficiency as the existing methodologies that use 3T3 cells. This 3-D culture system can be used to induce transdifferentiation of SESCs on limbal stromal cells. In embodiments where SESCs don't grow well on the membrane in this 3-D culture, we can seed the SESCs directly onto the growth-arrested human limbal stromal cells.

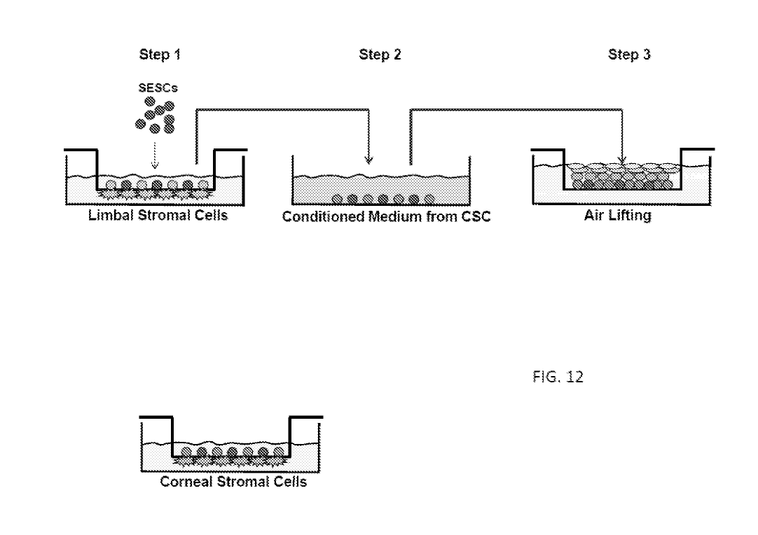

A challenge in this field is the lack of known specific marker(s) that can distinguish SESCs from LSCs. However, markers of mature epidermal (K10) and corneal epithelial cells (K12) are available. Therefore, we can use the ability to differentiate into mature corneal epithelial cells expressing K12 as an indicator for successful reprogramming. The isolated SESCs can be induced to transdifferentiate into the corneal lineage and then differentiate into mature corneal epithelial cells using a 3-step protocol (FIG. 12). Typically the SESCs are co-cultured onto the growth arrested limbal stromal cells for 5-14 days to induce transdifferentiation and generate induced LSCs (iLSCs, Step 1). Following this step, the epithelial cells are replated and cultured in conditioned medium from corneal stromal cells for additional 5-14 days to induce differentiation to mature corneal epithelial cells (Step 2). The second step likely further induces transdifferentiation of SESCs to iLSCs and induces differentiation of iLSCs. If this method does not induce differentiation efficiently, co-culture with the growth-arrested corneal stromal cells using the 3-D method can be used as the alternative approach. The third step involves air lifting to further induce maturation (Step 3). It is possible that co-culture with corneal stromal cells might not be sufficient to fully differentiate the reprogrammed epithelial cells. Air lifting is a well-established method in which culture medium is reduced to expose the very surface of the epithelial cells for 10 to 16 days to promote stratification and further differentiation (see, e.g. Koizumi et al., Graefes Arch Clin Exp Ophthalmol 2007; 245:123-134).

Phenotypic analysis using the two markers, K12 and K10 can be performed after each of the three steps. If the reprogramming is successful, the epithelial cells express the corneal epithelial marker K12 but not the epidermal epithelial marker K10 in Step 2 and 3. The length of co-culture will start at 5 days for the first two steps and will increase at 2-day increment up to 21 days if no K12 expression is detected after any of the three steps.

If K12 expression is induced, the optimal length of the co-culture that will produce the lowest percentage of K12+ cells in Step 1 with the highest K12 expression in Step 3 can be determined. The goal is to generate the undifferentiated, but not the differentiated iLSCs. In the event that no K12 expression is detected in Step 3 after maximal length of culture in Step 1 and 2, we can move on to the protocol discussed immediately below.

### Optimize the Transdifferentiation Protocol by Modulating Regulatory Pathways.

I. The Role of Dkk2 in SESCs Reprogramming.

As discussed above, Wnt/β-catenin and Notch pathways play pivotal roles in the fate determination decision between epidermal and corneal epithelial lineage in mice. In particular, Dkk2 is necessary for corneal development and activation of Notch1 is required to maintain the corneal lineage. Dkk2 alone or in combination with Notch1 activation may facilitate and increase the efficiency of the reprogramming process in humans. Functional recombinant Dkk2 protein can be provided by Dr. Jie Zheng (St. Jude Children's Research Hospital) (see, e.g. Lin et al., Proc Natl Acad Sci USA 2010; 107:4194-4199). It inhibits Wnt3a activities with an IC50 value around 8 nM in the Wnt reporter gene assay (see, e.g. Lujan et al., Proc Natl Acad Sci USA 2012; 109:2527-2532). Therefore, we can use the 3-step coculture protocol as described above and add Dkk2 to the culture medium at various concentrations starting at 8 nM to the Step 1 co-culture, which is the reprogramming step. Phenotypic analysis can be performed in each of the 3 steps to determine the outcome of transdifferentiation by the expression of K12 and K10 in the cultured epithelial cells. The optimal concentration of Dkk2 that achieves the highest portion of K12+ cells in Step 3 can be determined. This concentration of Dkk2 can be used. If reprogramming is still not successful, Dkk2 at 125 nM which would achieve 90% inhibition can be used instead.

II. The Role of Notch1 in SESC Reprogramming.

In parallel, we can investigate the role of Notch1 activation in the reprogramming process. Since only the Notch ligands, Dill and Jag1 are expressed in the corneal and limbal epithelial cells, we can add these two Notch ligands in Step 1, Step 2, or both Step 1&2. To activate Notch1 using these recombinant Notch ligands in Step 1, human limbal stromal cells overexpressing these ligands can be generated using a lentiviral vector that contains human DII1, Jag1 or both cDNAs. SESCs can be co-cultured with limbal stromal cells expressing Notch ligands or control cells not expressing ligands. To expose cells to these ligands in Step 2, culture dishes can be coated with soluble DII1, Jag1 or both ligands using the established protocol (FIG. 11B), respectively, and the epithelial cells from Step 1 can be subcultured onto the coated plates. If the epithelial cells do not grow well on the Notch ligand-coated dishes, corneal stromal cells expressing these Notch ligands can be generated in the similar fashion as in Step 1. The transdifferentiation efficiency can be determined by phenotypic analysis described above using K12 and K10. The optimal co-culture condition that generates the highest K12-expressing cells in Step 3 and lowest in Step 1 can be used in the subsequent experiments. In the event that no K12+ cells are generated, all different Notch ligand, namely DII1−, Jag1− and DII1/Jag1− expressing limbal and corneal stromal cells, can be used in the next step.

III. Possible Synergic Effect of Dkk2 and Notch1 in SESCs Reprogramming.

Next we can investigate whether combination of Dkk2 and Notch1 activation will promote the reprogramming of SESCs into LSCs. Dkk2 can be added to the co-culture with limbal and corneal stromal cells that express Notch ligand(s) in Step 1 and Step 2, respectively. The outcome of reprogramming and the optimal combination of Dkk2 and Notch1 activation for reprogramming can be investigated using the phenotypic analysis described above. The optimal condition of reprogramming can be used in the following experiments.

IV. Inhibition of Differentiation of iLSCs During Expansion.

To obtain a sufficient amount of iLSCs for transplantation, these iLSCs from Step 1 will likely need to be expanded in culture. This could be achieved by subculturing the iLSCs after Step 1 (expansion step). Differentiation of LSCs occurs during the in vitro expansion process (see, e.g. Nakatsu et al., Investigative Ophthalmology & Visual Science 2011; 52:4734-4741). A higher number of stem/progenitor cells transplanted into the LSCD eyes positively correlates with the long-term success of the graft in humans (see, e.g. Rama et al., N Engl J Med 2010; 363:147-155). Limbal specific Wnt molecules, such as Wnt6 could reduce the differentiation of LSCs in culture (FIG. 10). We can use limbal specific Wnt in the expansion step to prevent differentiation. We can use the same approach to introduce each of these four individual limbal Wnt in the culture and investigate which Wnt would mostly effectively reduce differentiation.

If the highest efficiency of the reprogramming is less than 100%, there can be epithelial cells still committed to the epidermal lineage in the co-culture and they would need to be removed to obtain a pure population of reprogrammed LSCs that could become suitable for transplantation in humans. To dissect which type of SESC has the capability to reprogram to LSC, clonal expansion of the freshly isolated single SESC can be carried out first. Half of each individual clone can be used in the reprogramming protocol under the optimal co-culture conditions elucidated above. The other half of the clone can be used for differential gene profiling using RNA-seq techniques to elucidate the molecular signature of those SESCs that have the capacity to be reprogrammed into LSCs by comparing to the gene profile of those SESCs that lack such capacity. Surface molecules can be selected as the potential signature candidates. If the expression at the protein level is confirmed using immunohistochemistry, such cell surface molecule(s) can be further investigated as to whether they can be used as biomarker(s) to separate live SESCs that have the maximal capacity to be reprogrammed from those SESCs that have less capacity.

Testing the Function of iLSCs in a LSCD Mouse Model.

The ability of these iLSCs to reconstruct a healthy ocular surface can be tested in a mouse model of LSCD using established methods (see, e.g. Meyer-Blazejewska et al., Stem Cells 2011; 29:57-66). To eliminate the risk of rejection and to enable long-term survival study (>2 months), nude mice can be used. The iLSCs can be seeded on fibrin gel for 2-3 days to generate a fibrin cell sheet. LSCD can be created in nude mice by removing the entire limbal and corneal epithelium using an Algerbrush II corneal rust ring remover. The fibrin cell sheet will then be transplanted and secured onto the denuded cornea of these mice using human fibrin glue (commercially available) and sutures. The control groups will include mice given fibrin gel containing primary SESCs, mice given fibrin gel alone, and mice that receive no transplant. The ability of the transplanted cells to reconstruct and maintain a normal corneal epithelial layer can be evaluated on the basis of the degree of neovascularization and transparency of the epithelial layer in comparison to the controls in 1 week and in 1, 2, 3 and 6 months. The establishment of LSCD can be confirmed in the control mice that do not receive any transplant. Both the survival of the transplanted iLSCs and whether they can retain their progenitor phenotype in vivo can be determined by the expression level of putative stem cell markers, ABCG2, ΔNp63α, and K15/14. Expression of K12 signifies the maturation of the iLSCs in vivo. Human cells can be easily distinguished from mouse cells by using an anti-human mitochondria antibody.

The foregoing description of one or more embodiments of the invention has been presented for the purposes of illustration and description. It is not intended to be exhaustive or to limit the invention to the precise form disclosed. Many modifications and variations are possible in light of the above teaching.

All publications, patents, and patent applications cited herein are hereby incorporated by reference in their entirety for all purposes. Those of skill in this art understand that aspects of this technology can be adapted to form a wide variety of embodiments of the invention. All literature and other references identified in this disclosure are incorporated herein by reference.

1. A system for culturing human limbal stem cells obtained from corneal epithelium comprising:
a container comprising a culture media for the human limbal stem cells;
a porous membrane disposed in the culture media so as to form a first cell culture compartment and a second cell culture compartment, wherein the porous membrane allows soluble factors to migrate between the first cell culture compartment and the second cell culture compartment while simultaneously preventing cells from migrating between the first cell culture compartment and the second cell culture compartment;
human limbal stem cells disposed in the first cell culture compartment;
human feeder cells disposed in the second cell culture compartment, wherein the mammalian feeder cells secrete one or more soluble factors that faciliate the growth of the human limbal stem cells.

2. The system of claim 1, wherein the mammalian feeder cells comprise at least one of: human bone marrow derived mesenchymal stem cells, human adipose derived mesenchymal stem cells, or human limbal fibroblasts.

3. The system of claim 1, wherein the system further comprises a sheet of a fibrin material disposed in the container.

4. The system of claim 1, wherein the porous membrane:
comprises a polyethylene terephthalate; and/or
comprises pores having a size less than 3 μm.

5. The system of claim 1, wherein the porous membrane is disposed is the system in a horizontal orientation.

6. The system of claim 1, wherein the first cell culture compartment and the second cell culture compartment are arranged in the system so that the mammalian feeder cells are below the human limbal stem cells.

7. The system of claim 1, wherein human limbal stem cells are:
scraped from limbal tissue; and/or
pretreated with a protease; and/or
pipetted so as to break cell sheets into clusters of cells;
prior to being placed in the first cell culture compartment.

8. A system for culturing human limbal stem cells comprising:
a container comprising a culture media for human limbal stem cells of the corneal epithelium;
a limbal tissue sample explant disposed in the culture media wherein the tissue sample explant comprises human limbal stem cells; and
human feeder cells disposed in the culture media at a location proximal to the human limbal stem cells so that soluble factors produced by the feeder cells migrate to the human limbal stem cells.

9. The system of claim 8, further comprising a porous membrane disposed in the culture media so as to form a first cell culture compartment and a second cell culture compartment, wherein the porous membrane allows soluble factors to migrate between the first cell culture compartment and the second cell culture compartment while simultaneously preventing cells from migrating between the first cell culture compartment and the second cell culture compartment.

10. A method of maintaining human limbal stem cells of the corneal epithelium in an undifferentiated human limbal stem cell phenotype, wherein the phenotype is characterized by a small, uniform and compact cellular morpology, the method comprising culturing the human limbal stem cell cells in the system of claim 1.

11. The method of claim 10, wherein the phenotype is characterized by observing expression of at least one of: ATP-binding cassette subfamily G member 2 (ABCG2), Δp63α, or stage-specific embryonic antigen-4 (SSEA4) in the human limbal stem cells.

12. The method of claim 10, wherein the method results in a rate of human limbal stem cells proliferation that is greater that a comparable rate of human limbal stem cell proliferation that is observed in methods that do not use the porous membrane.

13. A method of facilitating the proliferation of human limbal stem cells within cell clusters, the method comprising culturing the human limbal stem cells in the system of claim 1.

14. The method of claim 13, wherein the human limbal stem cells are disposed in the system at a location and in an orientation selected to influence the polarity of the cells.

15. A method of generating cells having a human limbal stem cell phenotype comprising:
(1) disposing at least one of hair follicle stem cells, skin epithelial stem cells, embyronic stem cells or induced pluripotent stem cells in the first cell culture compartment of the system of claim 1;
(2) disposing at least one of human limbal stromal cells or corneal stromal cells in the second cell culture compartment of the system of claim 1;
(3) allowing soluble factors to migrate from the cells in the second cell culture compartment to the first cells culture compartment, wherein the migration of the soluble factors results in the generation of human limbal stem cells from the hair follicle stem cells, epidermal stem cells, embyronic stem cells or induced pluripotent stem cells;
so that cells having a human limbal stem cell phenotype are generated.

16. The method of claim 15, wherein the human limbal stromal cells or corneal stromal cells have been treated with mytomycin C or radiation so as to induce growth arrest.

17. The method of claim 15, wherein skin epithelial stem cells are disposed in the first cell culture compartment.

18. The method of claim 15, wherein human limbal stromal cells are disposed in the second cell culture compartment.

19. The method of claim 15, wherein corneal stromal cells are disposed in the second cell culture compartment.

20. The method of claim 15, further comprising observing expression of at least one of: ATP-binding cassette subfamily G member 2 (ABCG2), Δp63α, or stage-specific embryonic antigen-4 (SSEA4) in the cells having the human limbal stem cell phenotype.
